(19) United States
(12) Patent Application Publication
(10) Pub. No.: US 2005/0137102 A1
(43) Pub. Date: Jun. 23, 2005

(54) Foamable industrial strength hand cleanser

(75) Inventors: V. Scott Shoaf, Alpharetta, GA (US); Ralph Solarski, Alpharetta, GA (US)

Correspondence Address: DORITY & MANNING, P.A., POST OFFICE BOX 1449, GREENVILLE, SC, 29602-1449, US

(73) Assignee: Kimberly-Ckark Worldwide, Inc.

(21) Appl. No.: 10/738,960

(22) Filed: Dec. 17, 2003

(51) Int. Cl. C11D 1/00; A61K 7/50

(52) U.S. Cl. 510/138

(57) ABSTRACT

A foamable industrial strength cleansing composition is disclosed. The cleansing composition includes a cleaning solvent, such as d-limonene in conjunction with a mixture of surfactants. The surfactants include foaming agents and emulsifiers. When mixed with air, the cleansing composition forms a foam that may be used to clean dirt, grease and grime from the hands of a user.

### BACKGROUND OF THE INVENTION

Conventional hand cleansing formulations contain solvents and/or surfactants capable of solubilizing or emulsifying soils present on the skin's surface. The cleaning products may come in the form of, for instance, a bar soap which produces a lather or foam by agitation with the hands in the presence of water or a gel that may be, for instance, pumped from a dispenser.

Recently, consumer foamable cleansers have appeared on the market. Foamable cleansers are typically kept in a dispensing container that mixes the cleansing composition with air when dispensed immediately forming a foam or lather. These types of products have been used extensively in the medical field by doctors and other medical personnel. Compositions dispensed as foams have been regarded as desirable in part because of an association of foam with cleaning ability.

Most of the foamable cleansers sold in the past have been formulated and designed to have mild cleaning properties. In particular, many of the compositions are advertised as being mild on the skin to prevent against overdrying and prevent the composition from removing natural fats that may be present on the skin. Some of the foamable compositions have also been touted for their ability to moisturize the skin.

In fact, foamable hand cleansers have not been formulated for use in industrial settings. There has been a perception in the past that foamed hand cleansers were not capable of being formulated to have sufficient strength capable of removing grease, grime and other hard-to-remove substances that are typically found in harsher environments.

As such, a need currently exists for a foamable hand cleanser that has industrial strength.

### SUMMARY OF THE INVENTION

In general, the present invention is directed to a foamable industrial strength hand cleanser. The foamable hand cleanser includes a cleaning solvent capable of removing heavy dirt and grime from the hands of a user. The cleaning solvent, for instance, in one embodiment is d-limonene. In other embodiments, the cleaning solvent may be a hydrocarbon solvent such as a dibasic ester, a terpene, a mineral spirit, a naphtha, other petroleum distillates, halogenated solvents, and the like.

In accordance with the present invention, the cleaning solvent is combined with a mixture of surfactants. The surfactants are added for various purposes. For instance, at least one surfactant is added as a foaming agent and is present in the hand cleanser in an amount sufficient for the cleanser to form a foam when aerated. For instance, the surfactant is added in an amount sufficient for the cleanser to form a foam or lather when pumped from a dispenser and mixed with air.

In one embodiment, two foaming agents are present in the cleanser and include an anionic surfactant and an amphoteric surfactant. The anionic surfactant may be, for instance, sodium laureth sulfate, while the amphoteric surfactant may be cocamidopropyl betaine.

The industrial strength hand cleanser also includes a surfactant that acts as an emulsifier. The emulsifier may be, for instance, an alkoxylated carboxylic acid, such as a fatty ester of a polyalkylene glycol, which is a nonionic surfactant. For example, in one embodiment, a PEG castor oil and a PEG oleate are present in the cleanser as emulsifiers. The emulsifiers are present in an amount sufficient to promote the formation and stabilization of an emulsion between the ingredients.

The foamable industrial strength hand cleanser further includes water. The water may be present in an amount of at least 20% by weight, such as from about 30% to about 80% by weight. In one particular embodiment, water is present in an amount from about 50% by weight to about 75% by weight.

In addition to an industrial strength cleaning solvent, one or more foaming agents, one or more emulsifiers, and water, the industrial strength hand cleanser may include various other additives as desired. For instance, pH adjusters, buffers, antioxidants and preservatives may be present in the cleanser. pH adjusters and buffers that may be present in the composition include, for instance, citric acid and sodium citrate. An antioxidant may be present to prevent oxidation of the cleaning solvent, especially when d-limonene is present. The antioxidant may be, for instance, a butylated hydroxytoluene (BHT).

Other features and aspects of the present invention are discussed in greater detail below.

### DETAILED DESCRIPTION OF THE INVENTION

The invention will now be described in detail with reference to particular embodiments thereof. The embodiments are provided by way of explanation of the invention, and not meant as a limitation of the invention. For example, features described or illustrated as part of one embodiment may be used with another embodiment to yield still a further embodiment. It is intended that the present invention include these and other modifications and variations as come within the scope and spirit of the invention.

The present invention is directed to an industrial strength foamable hand cleanser. Various foamable cleansers have been proposed in the past. The prior compositions, however, were limited to cleansers used by medical personnel to disinfect their hands or to very mild foamable cleansers that were designed to be used by consumers for everyday use. The foamable hand cleanser of the present invention, however, is designed for use in industrial settings for removing heavy dirt, grease, grime, and the like. The cleanser is also foamable meaning that the composition will form a foam or lather when aerated. For instance, the industrial strength cleanser may be contained in a dispenser that, when pumped, combines air with the cleanser composition for creating a foam.

The industrial strength cleanser of the present invention contains an industrial strength cleaning solvent combined with a mixture of surfactants. The surfactants may be anionic surfactants, nonionic surfactants, or amphoteric surfactants. The surfactants are selected and present in amounts so that the composition will foam when aerated and so that the composition will remain stable for a reasonable length of time. The surfactants chosen for inclusion in the composition include foaming agents and emulsifiers. Some surfactants, however, may serve multiple purposes when included in the composition.

In one embodiment, the cleaning solvent present in the hand cleanser is d-limonene. d-limonene contains 1-methyl-4′(1-methylethenyl)cyclohexene. d-limonene may be extracted from orange peels and from lemons and is practically insoluble in water.

Instead of or in addition to d-limonene, the hand cleanser may also contain other cleaning solvents, such as hydrocarbon solvents. Synthetic and natural hydrocarbon solvents may include dibasic esters, terpenes, mixtures of isoprenoid and mineral oil substances, naphthas, glycol ethers, parrafinic and isoparrafinic hydrocarbons, aromatic hydrocarbons, petroleum distillates, vegetable oils, animal oils, organic halides, halogenated solvents, and alcohols. Examples of dibasic esters may include dimethyl glutarate, dimethyl adipate, and dimethyl succinate.

The amount of cleaning solvent present within the foamable industrial strength hand cleanser depends on various factors. In general, higher amounts of cleaning solvents may prevent the formation of a foam or lather. In one embodiment, the cleaning solvent may be present in an amount from about 1% to about 8% by weight, such as from about 3% to about 6% by weight. In one particular embodiment, for instance, d-limonene is present in the hand cleanser in an amount of about 5% by weight.

As described above, the cleaning solvent is combined with various surfactants in order to create a foamable composition that has industrial strength. The surfactants that may be used in the present invention include those disclosed in U.S. Pat. No. 6,495,151, U.S. Pat. No. 6,211,139, U.S. Pat. No. 5,720,961, and U.S. Pat. No. 3,962,150, which are all incorporated herein by reference to the extent that they are consistent with the teachings of the present invention. In one particular embodiment of the present invention, at least one surfactant is selected that serves as a foaming agent, while at least one other surfactant is selected that serves as an emulsifier.

Foaming agents that may be used in the present invention include surfactants capable of causing the composition to foam when aerated. Most foaming agents that may be used in the present invention also serve as cleansing agents and therefore assist in cleaning the hands of a user when present. Many of the foaming agents also serve as emollients.

In one embodiment of the present invention, the industrial strength foamable hand cleanser includes two foaming agents. One foaming agent is an anionic surfactant, while the other foaming agent is an amphoteric surfactant. The anionic surfactant may be, for instance, an alkyl sulfate obtained by sulfating an alcohol having from about 8 to about 22 carbon atoms, such as from about 12 to about 16 carbon atoms. Other suitable anionic surfactants include alkyl benzene sulfonates in which the alkyl group contains from about 9 to about 15 carbon atoms, such as from about 11 to about 14 carbon atoms in a straight chain or a branched chain configuration; paraffin sulfonates having from about 8 to about 22 carbon atoms, such as from about 12 to about 16 carbon atoms in the alkyl moiety; olefin sulfonates having from about 8 to about 22 carbon atoms, such as from about 12 to about 16 carbon atoms; alkyl ether sulfates derived from ethoxylating an alcohol having from about 8 to about 22 carbon atoms, such as from about 12 to about 16 carbon atoms with from about 1 to about 30 moles of ethylene oxide, such as from about 1 to about 12 moles of ethylene oxide and then sulfonating; and alkyl glycerol ether sulfonates having from about 8 to about 22 carbon atoms, such as from about 12 to about 16 carbon atoms in the alkyl moiety.

In one particular embodiment, the anionic surfactant may be sodium laureth sulfate. Other examples, however, include ammonium lauryl sulfate, decyl polyglucose, ammonium cocoyl isothioniate and the like.

The anionic surfactant may be present in the cleanser in an amount from about 0.5% to about 10% by weight. For instance, the anionic surfactant may be present in an amount from about 1% to about 5% by weight. In one particular embodiment, the anionic surfactant is sodium laureth sulfate and is present in an amount from about 1% to about 2% by weight.

In addition to an anionic surfactant, the foamable industrial strength hand cleanser may also contain a foaming agent that comprises an amphoteric surfactant. The amphoteric surfactant may be, for instance, a betaine, which has been found to work particularly well with sodium laureth sulfate. Examples of suitable betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl)carboxymethyl betaine, stearyl bis-(2-hydroxyethyl)carboxymethyl betaine, oleyl dimethyl gamma-carboxypropyl betaine, lauryl bis-(2-hydroxypropyl)alphacarboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, amidobetaines, amidosulfobetaines, oleyl betaine (available as amphoteric Velvetex OLB-50 from Henkel), and cocamidopropyl betaine (available as Velvetex BK-35 and BA-35 from Henkel).

The amphoteric surfactant may be present in the hand cleanser in an amount from about 2% to about 15% by weight. In one particular embodiment, the amphoteric surfactant is cocamidopropyl betaine and the anionic surfactant is sodium laureth sulfate. In this particular embodiment, the cocamidopropyl betaine may be present in the foamable industrial strength hand cleanser in an amount from about 5% to about 10% by weight, such as from about 6% to about 8% by weight.

In addition to foaming agents, other surfactants may be present in the hand cleanser that serve as emulsifiers. The emulsifiers, for instance, maintain an emulsion between water present in the composition and the other ingredients, such as the cleaning solvent. The emulsifier may be, for instance, a nonionic surfactant. For example, the emulsifier may be an alkoxylated carboxylic acid, such as a fatty ester of a polyalkylene glycol. The polyalkylene glycol ether, for instance, may contain a fatty acid having a carbon chain of from about 12 carbon atoms to about 22 carbon atoms. Particular examples include PEG stearate, PEG distearate, PEG oleate, PEG castor oil (castor oil contains triglycerides of fatty acids), and the like. In the above compounds, polyethylene glycol may be present in an amount from about 2 moles to about 150 moles, such as from about 2 moles to about 12 moles.

In one particular embodiment of the present invention, the foamable hand cleanser includes a combination of PEG-2 castor oil and PEG-3 oleate. In this embodiment, each of the fatty esters of polyethylene glycol may be present in an amount from about 0.5% to about 10% by weight, such as from about 1% to about 4% by weight. For example, in one embodiment, the fatty esters of polyethylene glycol may be present in an amount of about 2% by weight.

In addition to serving as emulsifiers, alkoxylated carboxylic acids may also serve as emollients, solubilizers and suspending agents.

In addition to the above ingredients, the industrial strength foamable hand cleanser also includes water. The amount of water present in the cleanser may vary depending upon the particular application and the desired result. Water is generally present in the composition in an amount of at least 20% by weight, such as from about 30% to about 80% by weight. In one particular embodiment, for instance, water may be present in an amount from about 50% to about 75% by weight, such as from about 60% to about 70% by weight. Water may be added to the composition prior to or after shipping. For example, in one embodiment, a concentrated cleanser may be shipped to a site where water is later added.

The industrial strength foamable hand cleanser of the present invention may also contain minor amounts of various other additional ingredients to impart various desired characteristics to the composition. Examples of suitable additives include thickening agents, coloring agents, perfumes, preservatives, antiseptic agents, antibacterial agents, disinfectants, emollients, vitamins, foam stabilizers, humectants, and the like.

When the industrial strength hand cleanser contains d-limonene, in some applications, an antioxidant may be added in order to prevent the d-limonene from oxidizing. Antioxidants may include, for instance, butylated hydroxyanisole (BHA), citric acid, vitamin E, other tocopherol derivatives, and the like. In one particular embodiment, for instance, the cleanser may contain butylated hydroxytoluene (BHT) as an antioxidant. The antioxidant may be present in an amount from about 0.01% to about 3% by weight, such as from about 0.1% to about 0.5% by weight.

In order to maintain the cleansing composition within a desired pH range, the composition may also contain pH adjusters and buffers. For instance, in one embodiment, citric acid may be added in combination with a metal citrate, such as sodium citrate. Citric acid may be added in order to decrease the pH while the sodium citrate may be added in order to increase the pH. When added in combination, both ingredients serve as a buffer system.

For instance, in one embodiment, citric acid may be present in the composition in an amount from about 0.001% to about 1% by weight, such as from about 0.01% to about 0.05% by weight. Sodium citrate, on the other hand, may be present in the composition in an amount from about 0.01% to about 1% by weight, such as from about 0.1% to about 0.5% by weight.

Another ingredient that may be present in the industrial strength foamable hand cleanser is a preservative. Many different preservative agents are available and may be used in the composition. Preservatives that may be used in the present invention include parabens having from about 1 carbon to about 5 carbons, such as methylparaben or propylparaben. Other preservatives include phenoxyethanol, quaternary ammonium salts, 2-bromo-2-nitropropane-1,3-diol, methyidibromo, glutanitrile, propylene glycol, dimethylol dimethyl hydantoin, and the like. The amount of any given preservative or mixture thereof included in the composition depends upon its potency and stability. For instance, the preservatives may be present in the composition in an amount from about 0.001% to about 1% by weight.

The industrial strength foamable hand cleanser may be prepared in any conventional manner, e.g. by simply admixture of the components. For instance, in one embodiment, d-limonene may be combined first with an antioxidant. The antioxidant and the cleaning solvent may then be mixed with an emulsifier and a foaming agent. To this mixture, water and a buffer may be added. Finally, various preservatives may be added at the end in order to formulate the cleansing composition.

The foamable composition of the present invention is specifically formulated to form a foam when aerated. In this regard, the composition may be contained in a foam dispensing pump container comprising a container body in which the foamable composition is stored and a pump spray that may be, for instance, threadably attached to the container body. The container may have a volume of, for instance, one gallon or less. The foam dispensing pump container is of the type that entrains air in the foamable composition as it is expressed through the pump sprayer, thereby causing the composition to be delivered as a stable, dense foam or lather.

Various foam dispensing pump containers are known such as those disclosed in U.S. Pat. No. 5,271,530, U.S. Pat. No. 5,429,279, and U.S. Pat. No. 5,337,929, which are all incorporated herein by reference.

The present invention may be better understood with reference to the following example.

### EXAMPLE

The following industrial strength cleansing composition was formulated in accordance with the present invention. The ingredients listed in the table below were obtained from various commercial sources. The column marked “% in Formulation” refers to the amount of the commercial product incorporated into the formulation. The “% Active” column refers to the active concentration of the ingredient in the commercial product used. Thus, the “Actual % in Formulation” provides the actual amount of the ingredient present in the formulation.

TABLE 1

| | % in | | Actual % in |
|---|---|---|---|
| Ingredient | Formulation | % Active | Formulation |
| d-limonene | 5 | 95 | 4.75 |
| PEG-2 Castor Oil | 4 | 50 | 2.00 |
| PEG-3 Oleate | 4 | 50 | 2.00 |
| Sodium Laureth | 5 | 27 | 1.35 |
| Sulfate | | | |
| BHT | 0.125 | 100 | 0.13 |
| Water | 61 | 100 | 61.00 |
| Cocamidopropyl | 20 | 35 | 7.00 |
| Betaine | | | |
| Citric Acid | .02 | 100 | 0.02 |
| Sodium Citrate | .26 | 100 | 0.26 |
| Methyldibromo | <1 | | <1% |
| glutanitrile | | | |
| Phenoxyethanol | <1 | | <1% |
| Propylene Glycol | <1 | | <1% |
| DMDM Hydantoin | <1 | | <1% |
| Methyl Paraben | <1 | | <1% |

The emulsifers PEG castor oil and PEG oleate are commercially available as a blend under the trade name EMULSION BLEND DV 3317 sold by Rhodia. Sodium laureth sulfate is commercially available as STANDAMID ES-1 from Cognis. Methyidibromo glutanitrile and phenoxyethanol are present in a product sold under the trade name MERGAURD 1200 marketed by the Ondeo Nalco Company, while the preservatives propylene glycol, DMDM hydantoin and methyl paraben are sold together in a product called PARAGON marketed by the McItyre Group.

In formulating the above foamable cleanser, BHT was first dissolved in d-limonene. Thereafter, PEG castor oil and PEG oleate along with sodium laureth sulfate were added. To the above ingredients, water, cocamidopropyl betaine, citric acid and sodium citrate were added and the resulting mixture was mixed well. Thereafter, the preservatives were added to the composition.

The above foamable composition was placed in a pump-like dispenser which mixed the composition with air when pumped from the container. Upon pumping the container, a foam was produced that was found to have good cleaning strength.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A foamable, industrial strength hand cleanser comprising:
a cleaning solvent comprising a material selected from the group consisting of d-limonene, a hydrocarbon solvent, and mixtures thereof;
at least one foaming agent present in the hand cleanser in an amount sufficient for the cleanser to form a foam when aerated;
water present in an amount of at least about 20% by weight; and
at least one emulsifier present in an amount sufficient to promote the formation and stabilization of an emulsion between the cleaning solvent, the foaming agent, and the water.

2. A foamable industrial strength hand cleanser as defined in claim 1, wherein the cleaning solvent comprises d-limonene.

3. A foamable industrial strength hand cleanser as defined in claim 1, wherein the cleaning solvent is present in the hand cleanser in an amount from about 1% to about 8% by weight.

4. A foamable industrial strength hand cleanser as defined in claim 1, wherein the foaming agent comprises an anionic surfactant and an amphoteric surfactant.

5. A foamable industrial strength hand cleanser as defined in claim 4, wherein the anionic surfactant comprises an alkyl sulfate and the amphoteric surfactant comprises a betaine.

6. A foamable industrial strength hand cleanser as defined in claim 4, wherein the anionic surfactant comprises sodium laureth sulfate and the amphoteric surfactant comprises cocamidopropyl betaine.

7. A foamable industrial strength hand cleanser as defined in claim 1, wherein the emulsifier comprises an alkoxylated carboxylic acid.

8. A foamable industrial strength hand cleanser as defined in claim 1, wherein the emulsifier comprises a fatty ester of a polyalkylene glycol.

9. A foamable industrial strength hand cleanser as defined in claim 1, wherein the emulsifier comprises PEG castor oil and PEG oleate.

10. A foamable industrial strength hand cleanser as defined in claim 1, wherein water is present in an amount from about 50% to about 75% by weight.

11. A foamable industrial strength hand cleanser as defined in claim 1, wherein the hand cleanser further comprises an antioxidant.

12. A foamable industrial strength hand cleanser as defined in claim 1, wherein the hand cleanser further comprises a buffer.

13. A foamable industrial strength hand cleanser as defined in claim 1, wherein the hand cleanser further comprises a preservative.

14. A foamable industrial strength hand cleanser comprising:
a cleaning solvent comprising d-limonene;
at least two foaming agents present in the hand cleanser, the foaming agents comprising an anionic surfactant and an amphoteric surfactant;
water; and
at least one emulsifier present in an amount sufficient to promote the formation and stabilization of an emulsion between the cleaning solvent, the foaming agent and the water, the emulsifier comprising at least one fatty ester of a polyalkylene glycol.

15. A foamable industrial strength hand cleanser as defined in claim 14, wherein the anionic surfactant comprises a alkyl sulfate and the amphoteric surfactant comprises a betaine.

16. A foamable industrial strength hand cleanser as defined in claim 14, wherein the anionic surfactant comprises sodium laureth sulfate and the amphoteric surfactant comprises cocamidopropyl betaine.

17. A foamable industrial strength hand cleanser as defined in claim 15, wherein the anionic surfactant is present in an amount from about 0.5% to about 10% by weight and wherein the amphoteric surfactant is present in an amount from about 2% to about 15% by weight.

18. A foamable industrial strength hand cleanser as defined in claim 14, wherein the cleaning solvent is present in the hand cleanser in an amount from about 1% to about 8% by weight.

19. A foamable industrial strength hand cleanser as defined in claim 14, wherein the cleaning solvent is present in the hand cleanser in an amount from about 2% to about 6% by weight.

20. A foamable industrial strength hand cleanser as defined in claim 14, wherein the emulsifier comprises PEG castor oil and PEG oleate.

21. A foamable industrial strength hand cleanser as defined in claim 14, wherein the hand cleanser includes at least two fatty esters of a polyalkylene glycol, each fatty ester being present in an amount from about 0.5% to about 10% by weight.

22. A foamable industrial strength hand cleanser as defined in claim 14, wherein the hand cleanser further comprises a buffer.

23. A foamable industrial strength hand cleanser as defined in claim 22, wherein the buffer comprises citric acid and a citrate salt.

24. A foamable industrial strength hand cleanser as defined in claim 14, wherein the hand cleanser further comprises an antioxidant.

25. A foamable industrial strength hand cleanser as defined in claim 24, wherein the antioxidant comprises butylated hydroxytoluene.

26. A foamable industrial strength hand cleanser as defined in claim 25, wherein the antioxidant is present in the hand cleanser in an amount from about 0.1% to about 3% by weight.

27. A foamable industrial strength hand cleanser as defined in claim 14, wherein the hand cleanser further comprises a preservative.

28. A foamable industrial strength hand cleanser as defined in claim 27, wherein the preservative comprises a material selected from the group consisting of methyldibromo glutanitrile, phenoxyethanol, propylene glycol, dimethylol dimethyl hydantoin, methylparaben, and mixtures thereof.

29. A foamable industrial strength hand cleanser comprising:
a cleaning solvent comprising d-limonene;
water; and
a mixture of surfactants present in an amount sufficient to form a foamable emulsion when aerated, the mixture of surfactants comprising an alkyl sulfate, a betaine, and at least one fatty ester of a polyalkylene glycol.

30. A foamable industrial strength hand cleanser as defined in claim 29, wherein the alkyl sulfate comprises sodium laureth sulfate, wherein the betaine comprises cocamidopropyl betaine, and wherein the at least one fatty ester of a polyalkylene glycol comprises PEG castor oil and PEG oleate.

31. A foamable industrial strength hand cleanser as defined in claim 30, wherein the d-limonene is present in an amount from about 3% to about 6% by weight, wherein the alkyl sulfate is present in an amount from about 1% to about 5% by weight, wherein the betaine is present in an amount from about 5% to about 10% by weight, and wherein both the PEG castor oil and the PEG oleate are present in an amount from about 1% to about 4% by weight.

32. A foamable industrial strength hand cleanser as defined in claim 31, wherein water is present in the hand cleanser in an amount from about 50% to about 75% by weight.

33. A foamable industrial strength hand cleanser as defined in claim 30, wherein the hand cleanser further comprises an antioxidant, a buffer, and a preservative.

34. A foamable industrial strength hand cleanser as defined in claim 33, wherein the antioxidant comprises butylated hydroxytoluene and the buffer comprises citric acid and sodium citrate.

35. A dispensing container containing the foamable industrial strength hand cleanser as defined in claim 29, the dispensing container including a manual pump that converts the hand cleanser into a foam when manually operated.
